(19) United States
(12) Patent Application Publication
(10) Pub. No.: US 2005/0043288 A1
(43) Pub. Date: Feb. 24, 2005

(54) Composition and method for marking procaine penicillin

(76) Inventor: Thomas Tobin, Lexington, KY (US)

Correspondence Address: KING & SCHICKLI, PLLC, 247 NORTH BROADWAY, LEXINGTON, KY, 40507, US

(21) Appl. No.: 10/924,331

(22) Filed: Aug. 23, 2004

Related U.S. Application Data
(60) Provisional application No. 60/496,750, filed on Aug. 21, 2003.

(51) Int. Cl. A61K 31/43; C07D 499/48

(52) U.S. Cl. 514/192; 540/331

(57) ABSTRACT

A chemically marked procaine penicillin is provided as an antibiotic which can be distinguished from procaine used for anesthetic purposes. A preferred embodiment provides a procaine penicillin molecule wherein the procaine portion of the molecule comprises one or more substituted deuterium atoms in place of hydrogen atoms typically found in procaine penicillin. The resulting non-radioactive isotope can be easily identified via mass spectrometry as being distinct from any naturally occurring isotope, without negatively impacting the pharmacological nature of the drug itself. An alternative embodiment of the invention provides a method of marking the procaine penicillin through the substitution of various other isotopes in place of their naturally occurring counterparts. Methods of detection of the marked procaine penicillin molecule are also provided.

### REFERENCE TO RELATED APPLICATION

This application claims the benefit of priority in U.S. Provisional Application Ser. No. 60/496,750, filed on Aug. 21, 2003.

### FIELD OF INVENTION

The present invention is generally directed for use in the veterinary and pharmacological treatment of competitive animals. In particular, the invention provides a method for chemically marking procaine penicillin with atomic isotopes not otherwise found in nature. More particularly, a method of distinguishing between procaine penicillin administered therapeutically and procaine administered for use as an anesthetic is presented.

### BACKGROUND OF INVENTION

Procaine penicillin, e.g. Procaine Penicillin G (PPG) is an inexpensive antibiotic with high efficacy in the treatment of a variety of infectious diseases in animals. As a result, procaine penicillin has become the antibiotic of choice by veterinarians for the treatment of numerous illnesses.

Unfortunately, the drug's therapeutic use in the competitive racing and performance communities is restricted. Veterinarians are not able to administer procaine penicillin in the weeks immediately preceding a competition because post-race urine and/or blood tests are unable to distinguish between procaine administered as procaine penicillin for therapeutic reasons and procaine used solely for its anesthetic, performance-enhancing properties. Unable to determine the purpose of application, the procaine residue of either compound is uniformly treated as an illegal administration and generally results in significant sanctions against the owner/trainer of the animal.

Similar problems occur when meat processed with procaine penicillin is fed to competitive animals such as Greyhounds. The procaine residue from the ingested meat can be detected in the post-race urine and/or blood tests administered to the dogs. Current tests are unable to distinguish between procaine used to treat infections in the feed animals from procaine used as a local anesthetic to enhance the racing abilities of the dog. Unable to make this distinction, the racing community is forced to punish indiscriminately, all owners and trainers of any dog found with traces of procaine reside.

As such, a therapeutic form of procaine penicillin which is easily distinguished from its anesthetic counterpart will have a significant impact on the treatment and well being of competitive animals. Thus there is a need in the art for a form of procaine penicillin that is easily distinguished from procaine used as an anesthetic. No longer would veterinarians have to depend on less effective methods of treatment in the weeks preceding competitions. The ability to distinguish between procaine used therapeutically and procaine used for its anesthetic value is a long felt need in the art which would allow veterinarians to continue with the proper treatment of infectious diseases regardless of upcoming competitions. Likewise, Greyhound owners and trainers can feed meat that has been treated with procaine penicillin to their dogs without the fear of getting a positive blood or urine test from the procaine residue found in the meat.

Deuterium, a Hydrogen isotope with an aliquot of neutrons and protons in its nucleus, has been used in the pharmaceutical industry for many years. Until this point, its primary use has been to alter the pharmacological effect of the drug to which it has been substituted. Deuterated benzylpenicillins have been synthesized to increase their relative antibiotic potency. Biosynthesis of Deuterated Benzylpenicillins, Journal of Pharmaceutical Science, May 1973, at 856-7. Similarly, the metabolic rate of sevoflurane has been lowered by the substitution of deuterium for certain hydrogen atoms. U.S. Pat. No. 5,391,579. Additionally, the efficacy of nifedipine has been increased through deuteration. U.S. Pat. No. 5,846,514.

Stable isotopes and even long-lived radioisotopes of certain elements have been used to tag antibodies (or antigens). However, these isotopes were used for the determination of antigen (or antibody) concentrations by immunoassay analysis. In addition, deuterium was not listed as a viable isotope. U.S. Pat. No. 4,022,876. Moreover, deuterated compounds have been found beneficial in nonpharmaceutical ways. Deuterium is easily identified through mass spectrometry. Thus, deuterated compounds including deuterated procaine have been used to more easily observe the molecular behavior of the non-deuterated isotope. Multiple Binding Sites for Local Anesthetics in Membranes, Canadian Journal of Biochemistry, October 1980, at 986-95. Specifically deuterated organic compounds have been used as a proof of sender in the shipping industry. U.S. Pat. No. 5,474,937. Deuterated drugs have also been used in conjunction with their naturally occurring isotope in the drug rehabilitation industry. There, specific ratios of deuterated to non-deuterated drugs are administered to recovering addicts. The ratio can be monitored via mass spectrometry of urine samples to show that the addict is not supplementing his portions of the drug. U.S. Pat. No. 4,223,004.

To date, the use of deuterated compounds for the purpose of distinguishing a therapeutic drug from a similar drug used for illicit purposes, particularly in the competitive animal community, has not been described.

### SUMMARY OF THE INVENTION

A primary object of the present invention is to provide a novel formulation comprised of procaine penicillin such that the procaine portion of the compound is chemically distinguishable from procaine used as an anesthetic. In one embodiment, the invention provides a method of distinguishing procaine penicillin from procaine used as an anesthetic through the substitution of deuterium atoms for hydrogen atoms on at least one and not more than eighteen of the twenty hydrogen atoms found on the procaine molecule. Any hydrogen atom of the procaine molecule, along with any combination thereof, except those two hydrogen atoms on the benzylamino group may be substituted. The substituted deuterium atoms allow the procaine used in conjunction with the therapeutic use of procaine penicillin to be easily distinguished from procaine used as a local anesthetic and/or from the non-deuterated form of procaine penicillin. The ease and certainty with which the deuterated procaine can be distinguished from its non-deuterated isotope will allow the use of procaine penicillin in those previously discussed ways which are currently restricted.

### BRIEF DESCRIPTION OF THE DRAWINGS

FIG. 1 is a schematic representation showing the chemical formula for a Procaine molecule. The compound of FIG. 1 is commonly used as a local anesthetic. For this reason, the competitive animal community has restricted its use and any animal showing a positive test for procaine in its post-race-urine and/or blood sample will be sanctioned.

FIG. 2 is schematic representation showing the chemical formula of Procaine-d18. This compound is identical to the procaine depicted in FIG. 1 except that eighteen of its hydrogen atoms have been substituted with deuterium.

FIG. 3 is a schematic representation showing the chemical formula for Procaine Penicillin G. PPG is currently the most common form of penicillin administered to the competitive animal community. Unfortunately, the procaine portion of the compound and the residue thereof cannot be distinguished from procaine used for anesthetic purposes, limiting the PPG's use in the weeks immediately preceding races.

FIG. 4 is a schematic representation showing the chemical formula of one embodiment of the invention, Procaine-d18 Penicillin G. The deuterium atoms in this compound make it easy to distinguish from the procaine found in FIGS. 1 and 3 when examined via mass spectrometry.

FIG. 5 is a schematic representation showing the chemical formula of a presently preferred embodiment of the invention, Procaine-d5 Penicillin G. As seen, all five of the hydrogen atoms on one of the ethyl groups of the N,N Diethylamino functional group have been substituted with deuterium.

FIG. 6 is a schematic representation showing the chemical formula for a Procaine molecule wherein the eighteen (18) possible hydrogen atoms available for substitution with deuterium atoms are shown as R₁-R₁₈. The compound of FIG. 1 is commonly used as a local anesthetic. Substitution of the hydrogen atoms with one or more deuterium atoms at positions R₁-R₁₈ of the procaine molecule will sufficiently mark the procaine molecule and thereby differentiate the substituted molecule from the naturally occurring procaine molecule commonly used as a local anesthetic.

### DETAILED DESCRIPTION OF THE INVENTION

Additional objects, advantages and other novel features of the invention will be set forth in the description that follows and in part will become apparent to those skilled in the art upon examination of the foregoing or may be learned with the practice of the invention.

The present invention provides a composition and a method of distinguishing compositions comprised of procaine that are used for therapeutic reasons, e.g., the antibiotic procaine penicillin G (PPG), from compositions used as a local anesthetic, e.g., procaine. The ability to make such a distinction is particularly applicable in the competitive animal industry where a current inability to distinguish between the alternate uses of procaine has resulted in a complete restriction of its use. As a result of the restriction, veterinarians are currently unable to administer procaine penicillin, e.g. PPG, an effective antibiotic, in the weeks immediately preceding a race.

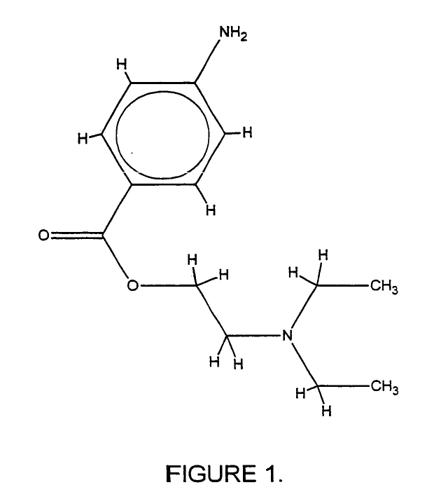

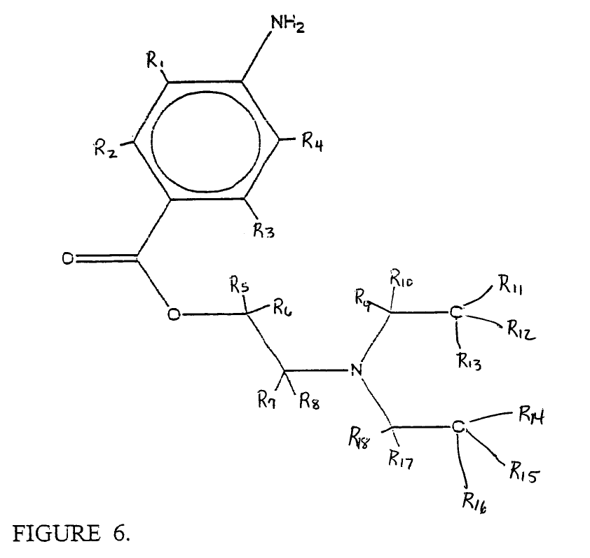

One embodiment of the method for distinguishing procaine used therapeutically from procaine used anesthetically involves the substitution of a deuterium atom for at least one of the hydrogen atoms found on the procaine molecule shown in FIG. 1. For example, FIG. 6 is a schematic representation showing the chemical formula for a procaine molecule wherein the eighteen (18) possible hydrogen atoms available for substitution with deuterium atoms are shown as R functional groups, namely R₁-R₁₈. In one presently preferred embodiment of the invention, a distinguishable deuterium-substituted procaine penicillin G is provided which is comprised of a substituted procaine portion of the PPG molecule wherein at least one of the functional R groups of the procaine portion of the molecule (denoted as R₁-R₁₈) is substituted with a deuterium atom.

The compound of FIG. 1 is commonly used as a local anesthetic. Substitution of the hydrogen atoms normally found at positions R₁-R₁₈ (shown in FIG. 6) with one or more deuterium atoms at positions R₁-R₁₈ of the procaine molecule will sufficiently mark the procaine molecule and thereby differentiate the substituted molecule from the naturally occurring procaine molecule commonly used as a local anesthetic which naturally contains no deuterium. Thus, in one embodiment, the invention provides a composition comprised of a procaine molecule as set forth in figure six (6) wherein in the “R” functional group represented by R₁-R₁₈, “R” is selected from the group consisting of hydrogen and deuterium and mixtures thereof.

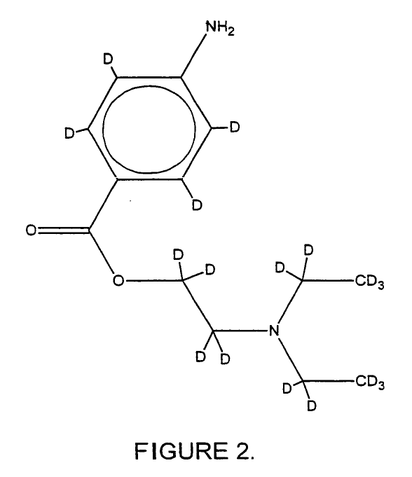

As depicted in FIG. 2, the fully deuterated form of procaine, Procaine-d18, is identical to its naturally occurring isotope except that eighteen of the twenty hydrogen atoms have been substituted with deuterium. Since mass spectrometry can easily distinguish between the two isotopes of procaine, the substituted deuterium atoms serve as chemical indicators that the deuterated compound is not naturally occurring.

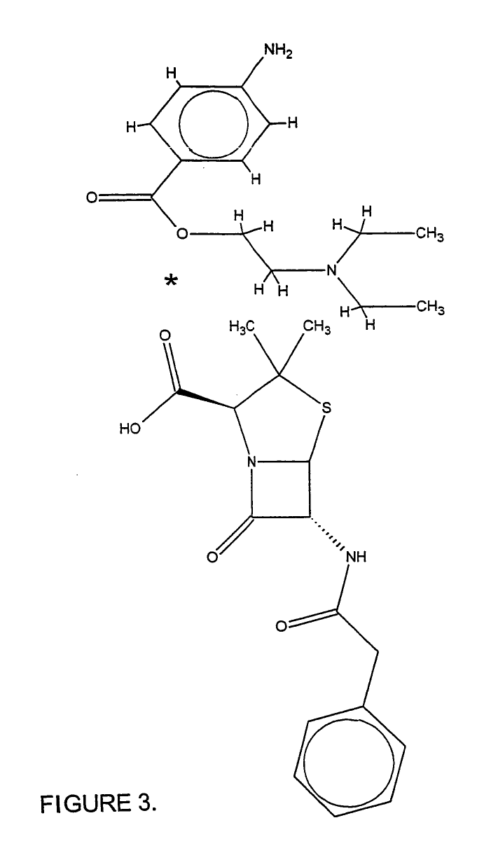

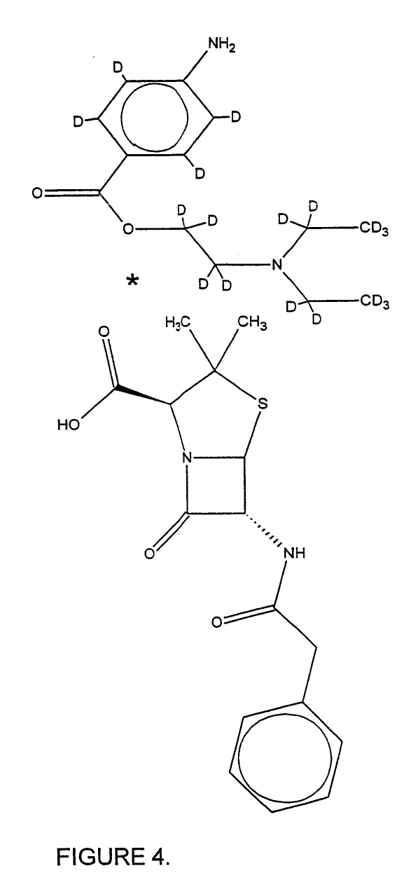

When a deuterium-substituted procaine, e.g., as shown in FIG. 2, is substituted for the procaine traditionally bonded to penicillin, as shown in FIG. 3, the resulting composition is a deuterated form of procaine penicillin, as depicted in FIG. 4. The penicillin salt shown in FIG. 4 has substantially similar pharmacological properties to its non-deuterated isotope, but is distinguishable by mass spectrometry. As such, the therapeutic benefits of the antibiotic can be reaped without the risk of confusion with any anesthetic use of procaine.

Although FIG. 2 depicts one embodiment of the invention, Procaine-d18, one of skill in the art will recognize that other variations are possible and are within the scope of the invention. In particular, the invention shall include procaine in which at least one (Procaine d1), preferably five (Procaine d-5) or ten (Procaine d-10) and not more than eighteen of the hydrogen atoms have been substituted with deuterium.

Thus, the methods of distinction provided by the invention are meant to include all forms of deuterated procaine penicillin, ranging from Procaine-d1 to Procaine-d18 and all manifestations thereof. Any combination of the procaine portion of the compound's eighteen hydrogen atoms that are not part of the benzylamino group can be substituted with deuterium while remaining within the scope of the present invention.

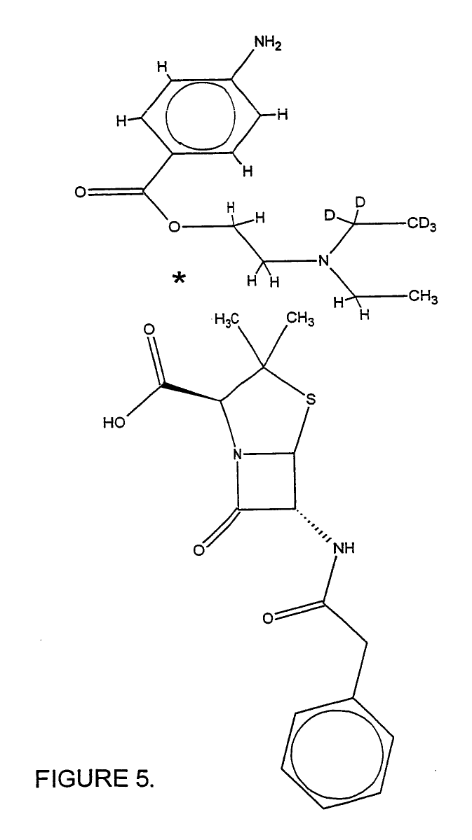

In one embodiment of the invention, the composition comprises a deuterated procaine penicillin salt comprised of procaine in which at least four of the exchangeable hydrogen atoms have been substituted with deuterium. As depicted in FIG. 5, a presently preferred embodiment of the composition comprising a deuterated procaine penicillin salt wherein five hydrogen atoms on a selected one of the two ethyl groups of the N,N Diethylamino functional groups of the procaine portion of the molecule have been substituted with deuterium. One of skill in the art can appreciate that substitution of five hydrogen atoms on a selected one or both of the ethyl groups on the N,N Diethylamino group is possible. Moreover, partial substitution of the hydrogen atoms on one or both ethyl groups is functionally equivalent. In a preferred embodiment of the invention the procaine portion of the procaine penicillin salt comprises 10 deuterium atom substitutions of the ethyl groups on the N,N Diethylamino group having the name: benzoic acid, 4-amino-2-(diethyl-d₁₀-amino)ethyl ester and the molecular formula C₁₃H₁₀D₁₀N₂O₂*HCl.

One embodiment of the invention provides a composition comprised of a procaine penicillin G molecule wherein the procaine portion of the molecule (procaine shown in FIG. 1) comprises the molecular formula C₁₃HₓD_{y}N₂O₂*HCl, wherein “D” is a deuterium atom; wherein “y” is the number of deuterium atoms and may vary between 1 and 18; and “x” is the number of hydrogen atoms represented by the formula 20−y. One of skill in the art can appreciate that given the teachings set forth herein, the number of possible hydrogen atoms available for substitution with deuterium is eighteen (18) as shown as the R functional groups shown as R₁-R₁₈ in FIG. 6. Thus, the number of hydrogen atoms represented in the above formula as “x” can vary between 2 and 20 depending upon the number of deuterium atoms substituted at R₁-R₁₈.

The commercial availability of deuterated procaine is hindered by its complex synthesis process and high cost of production. Accordingly, deuterated procaine is not readily available for use as an anesthetic. Thus, if the compositions provided by the invention comprised of deuterated procaine penicillin are used in the therapeutic treatment of animals, then any trace of the naturally occurring non-deuterated isotope of procaine in a post competition blood or urine sample would be a positive indicator that the procaine had been used as a performance enhancing anesthetic. Post race-urine and/or blood tests on competitive animals could easily distinguish between the deuterated and non-deuterated isotopes of procaine and thereby it would be possible to accurately infer the administrator's intended purpose of use.

Another embodiment of the method for distinguishing procaine used therapeutically from procaine used anesthetically involves the substitution of other isotopes and/or other combinations of isotopes to replace the naturally occurring elements of procaine. Two examples of isotopes that can readily serve as substituents are Carbon-13 and Nitrogen-17. By substituting either of these, any other, or any combination of other isotopes in place of their naturally occurring equivalents, the procaine used in conjunction with procaine penicillin could be marked and detected by mass spectrometry.

The foregoing description of preferred embodiments of the invention have been presented for purposes of illustration and description. They are not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A composition comprised of a procaine penicillin G molecule wherein the procaine portion of the molecule comprises the molecular formula C₁₃HₓD_{y}N₂O₂*HCl,
wherein “D” is a deuterium atom;
wherein “y” is the number of deuterium atoms and may vary between 1 and 18; and
wherein “x” is the number of hydrogen atoms represented by the formula 20−y.

2. The composition of claim 1, wherein “y” is 5; and “x” is 15.

3. The composition of claim 1, wherein “y” is 10; and “x” is 10.

4. A composition comprised of a procaine penicillin G molecule wherein the procaine portion of the molecule comprises the molecular formula C₁₃H₅D₁₅N₂O₂*HCl.

5. Composition comprised of a procaine penicillin G molecule wherein the procaine portion of the molecule comprises the molecular formula C₁₃H₁₀D₁₀N₂O₂*HCl.

6. A composition comprised of a procaine molecule as set forth in figure six (6) wherein in the “R” functional group represented by R₁-R₁₈, “R” is selected from the group consisting of hydrogen and deuterium and mixtures thereof.

7. The composition of claim 6, wherein R₁-R₈ are hydrogen and R₉-R₁₈ are deuterium.

8. The composition of claim 6, wherein R₁-R₁₃ are hydrogen and R₁₄-R₁₈ are deuterium.

9. The composition of claim 6, wherein R₁-R₈ are hydrogen, R₁₄-R₁₈ and R₆-R₁₃ are deuterium.

10. A method of differentiating between a naturally occurring procaine molecule as set forth in FIG. 1 and a deuterated procaine penicillin G molecule in a sample comprising detecting the presence of the molecule of claim 1 in the sample.

11. The method of claim 10, wherein the sample is a sample selected from the group selected from whole blood, serum, urine and saliva.

12. The method of claim 11, wherein the sample is from an animal selected from the group consisting of a dog or a horse.

13. The method of claim 12, wherein the animal is a racing animal and sample is a post race sample.
